(19) United States
(12) Patent Application Publication
(10) Pub. No.: US 2026/0118761 A1
(43) Pub. Date: Apr. 30, 2026

(54) COMPOUND FOR PHOTOLITHOGRAPHIC MEDIUM COMPOSITION, POLYMER, AND PHOTOLITHOGRAPHIC MEDIUM COMPOSITION

(71) Applicant: TAN KAH KEE INNOVATION LABORATORY, Xiamen, Fujian (CN)

(72) Inventors: Aiqiang ZHANG, Xiamen, Fujian (CN); Jialong WU, Xiamen, Fujian (CN); Renzhi CHEN, Xiamen, Fujian (CN); Yimin JIANG, Xiamen, Fujian (CN); Dong SHI, Xiamen, Fujian (CN)

(73) Assignee: TAN KAH KEE INNOVATION LABORATORY, Xiamen, Fujian (CN)

(21) Appl. No.: 19/165,803

(22) PCT Filed: Mar. 27, 2023
(86) PCT No.: PCT/CN2023/084158

§ 371 Date: Sep. 16, 2025

(30) Foreign Application Priority Data
Mar. 16, 2023 (CN) 202310256923.2

(51) Int. Cl. G03F 7/038; C08F 134/02

(52) U.S. Cl. CPC G03F 7/0384; C08F 134/02

(57) ABSTRACT

A compound and a polymer for a photolithographic medium composition, and the photolithographic medium composition is provided. The polymer has a structural unit represented by general formula (1) below. The compound and polymer are configured for use in an etching-resistant medium layer.

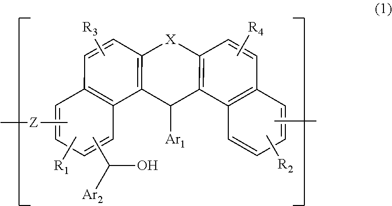

### FIELD

The present disclosure relates to a compound for a photolithographic medium composition, a polymer, and the photolithographic medium composition.

### BACKGROUND

The lithographic process is one of the most important processes in manufacturing semiconductor integrated circuit chips. Specifically, the lithographic process transfers the fine patterns of the integrated circuit on the mask plate to the photoresist by utilizing the light-sensitive function of photoresist. Then the final pattern is formed on the substrate medium by a subsequent etching process, or ion implantation is achieved by an ion implantation process. With the development of the semiconductor integrated circuit industry, integrated circuit patterns prepared by the lithographic process have become increasingly finer, evolving from tens of nanometers to several nanometers. In typical lithographic process techniques, the photoresist forms a photolithography pattern after exposure and development, and acts as a mask for etching the underlying substrate material. Therefore, it is required that the photoresist layer must have a certain degree of etching resistance. In addition, as the lithographic process develops and the requirements for finer patterns, the thickness of the photoresist layer had to be reduced to achieve better resolution, resulting in the photoresist alone could not fully function as mask. Therefore, a multi-layer stacking process (for example, photoresist-anti-reflective interlayer-etching resistant interlayer-substrate material layer) must be designed to meet the needs for producing the pattern with a high aspect ratio. After forming a photolithography pattern on the photoresist layer, the pattern can be transferred layer by layer to various intermediate layers and substrate material layer by utilizing the different etching rate of the etching gas on different material layers. In this process, the material in the upper layer acts as the etch mask for the material in the next layer.

In addition, when producing thin films of materials, the spin coating is cheaper than chemical vapor deposition (CVD) process, and the interlayers formed by the spin coating has better filling and planarizating properties. For this reason, the material needs to have a certain degree of solubility. Therefore, the material of the intermediate medium layers must have good etching resistance, planarizating and anti-reflection properties while taking into account the solubility of the material. Therefore, there is still a need in this field for the photolithographic medium materials with improved etching resistance and consideration of solubility.

### SUMMARY

The present disclosure relates to a polymer for a photolithographic medium composition, having a structural unit represented by general formula (1) below

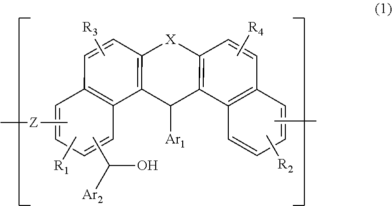

wherein X is a heteroatom, and each independently selected from the group consisting of an oxygen atom, a sulfur atom, and a nitrogen atom;
Ar₁ and Ar₂ are the same or different, and each independently selected from the group consisting of a C6-C20 aryl substituted by 0 to 3 R^{A}, a heteroaryl containing 3 to 20 skeletal ring-forming atoms and containing one or more identical or different heteroatoms substituted by 0 to 3 R^{A}, a heterocyclyl containing 3 to 20 skeletal ring-forming atoms and containing one or more identical or different heteroatoms substituted by 0 to 3 R^{A}, and a cyclic hydrocarbon group containing 3 to 20 carbon atoms substituted by 0 to 3 R^{A};
R₁ and R₂ are each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group;
R₃ and R₄ are each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group;
Z is selected from the group consisting of a single bond, a C1-C10 alkylene substituted by 0 to 3 R^{A}, a C6-C20 arylene substituted by 0 to 3 R^{A}, a C6-C20 aralkylene substituted by 0 to 3 R^{A}, a C4-C20 heteroaralkylene substituted by 0 to 3 R^{A}, and a C1-C10 heteroalkylene substituted by 0 to 3 R^{A};
R^{A} is each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group;
R¹¹ and R¹² are each independently selected from the group consisting of hydrogen, a C1-C8 alkyl, a C2-C8 alkenyl and an C2-C8 alkynyl.

The present disclosure also relates to a compound for a photolithographic medium composition, having a structural formula represented by general formula (11) below

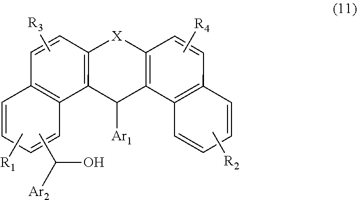

wherein X is a heteroatom, and independently selected from the group consisting of an oxygen atom, a sulfur atom, and a nitrogen atom;
Ar₁ and Ar₂ are the same or different, and each independently selected from the group consisting of a C6-C20 aryl substituted by 0 to 3 R^{A}, a heteroaryl containing 3 to 20 skeletal ring-forming atoms and containing one or more identical or different heteroatoms substituted by 0 to 3 R^{A}, a heterocyclyl containing 3 to 20 skeletal ring-forming atoms and containing one or more identical or different heteroatoms substituted by 0 to 3 R^{A}, and a cyclic hydrocarbon group containing 3 to 20 carbon atoms substituted by 0 to 3 R^{A};
R₁ and R₂ are each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group;
R₃ and R₄ are each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group;
R^{A} is each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group;
R¹¹ and R¹² are each independently selected from the group consisting of hydrogen, a C1-C8 alkyl, a C2-C8 alkenyl and a C2-C8 alkynyl.

The present disclosure also relates to a photolithographic medium composition that includes an acid generating agent, a crosslinking agent, and a medium material, wherein the medium material is the polymer and/or the compound described above in the present disclosure.

The present disclosure also relates to a photolithographic medium layer formed from the photolithographic medium composition of the present disclosure.

The compound and the polymer of the present disclosure have excellent performance in the aspects of solubility and etching resistance, the solubility of the compound and the polymer are optimized while the etching resistance is considered, and the compound and the polymer are very suitable as an etching-resistant medium material.

### BRIEF DESCRIPTION OF THE FIGURES

FIG. 1 shows ¹H-NMR spectrum for intermediate a-1.

### DETAILED DESCRIPTION

The present disclosure is described in further detail below in the drawings and embodiments. Through these explanations, the features and advantages of the present disclosure will become clearer and more apparent.

The wording “exemplary” is used herein to mean “used as an example, embodiment, or illustration”. Embodiments described herein as “exemplary” need not be construed as better or superior to other embodiments. The drawings illustrate various aspects of the embodiments, but it is not necessary to draw the drawings to scale unless otherwise noted.

Furthermore, the technical features involved in the different embodiments of the disclosure described below can be combined with each other as long as they do not contradict each other.

### Definitions

It is to be understood that the foregoing brief description and the following detailed description are exemplary and for illustrative purposes only and do not limit the subject matter of the invention in any way. It is important to note that the singular form used in this specification and claims includes the plural form of those referred to, unless otherwise explicitly stated in the text. It should also be noted that the terms “or” and “alternatively” are used to denote “and/or”, unless otherwise indicated. The use of the term “including” and other forms, such as “comprising”, “contain”, “containing”, is not restrictive.

When substituents are represented by conventional chemical formulas written from left to right, the substituents include the chemically equivalent substituents that would result if the structure were written from right to left as well. For example, CH₂O is equivalent to OCH₂.

The term “substituted or unsubstituted” includes both “substituted” and “non-substituted”, wherein “substituted” means that one or more hydrogen atoms on a particular atom has been substituted by a substituent, provided that the valence of the particular atom is normal and the substituted compound is stable, and “unsubstituted” means that the hydrogen atom on a particular atom has not been replaced. For example, substituted or unsubstituted ethyl (for example, if the substituent is a halogen) includes unsubstituted (—CH₂CH₃), monosubstituted (for example, —CH₂CH₂F), multisubstituted (for example, —CHFCH₂F, —CH₂CHF₂, etc.), or completely substituted (—CF₂CF₃). Those skilled in the art will understand that for any group consisting of one or more substituents, no substituent or pattern of substitution will be introduced which is not possible to exist spatially and/or synthesize. When the substituent is an oxo group (i.e., ═O), it means that two hydrogen atoms on the same atom are substituted.

When a variant (for example, R) occurs more than once in the composition or structure of a compound, its definition in each case is independent. Thus, for example, if a group is substituted by 0 to 3 R^{A}, the group may optionally be substituted by up to 3 R^{A}s, and there are independent alternatives for R^{A} in each case. In addition, combinations of substituents and/or variants thereof are allowed only if such combinations result in stable compounds. The term “optional” or optionally” means that the subsequent event or situation described may or may not occur, and that the explanation includes both the occurrence and non-occurrence of the event or situation.

Cm to Cn as used herein means that there are from m to n carbon atoms in the portion. As an example, a “C1-C8” group is a group having 1-8 carbon atoms in the portion, i.e., the group contains 1 carbon atom, 2 carbon atoms, 3 carbon atoms . . . 8 carbon atoms. Thus, for example, “C1-C8 alkyl” means an alkyl group having 1 to 8 carbon atoms, i.e., the alkyl group is selected from methyl, ethyl, propyl, isopropyl, n-butyl, isobutyl, sec-butyl, tert-butyl . . . octyl, etc. As used herein, numerical ranges, such as “1 to 8” refer to each integer within a given range. For example, “1 to 8 carbon atoms” means that the group may have 1 carbon atom, 2 carbon atoms, 3 carbon atoms, 4 carbon atoms, 5 carbon atoms, 6 carbon atoms, 7 carbon atoms, or 8 carbon atoms.

The term “alkyl” refers to an optionally substituted straight chain or an optionally substituted branch-chain saturated aliphatic hydrocarbon group, which is bound to the rest of the molecule by a single bond. As used herein, an alkyl may have 1 to 8 carbon atoms, for example, 1 to 6 carbon atoms, or 1 to 4 carbon atoms, or 1 to 3 carbon atoms. Examples of alkyl herein include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, tert-butyl, n-pentyl, iso-pentyl, neopentyl, tert-pentyl, hexyl, etc., and longer chain alkyl groups such as heptyl and octyl, etc. Where a group as defined herein, for example, “alkyl” appears in a numerical range, for example, “C1-C8 alkyl” means an alkyl group which can be composed of 1 carbon atom, 2 carbon atoms, 3 carbon atoms, 4 carbon atoms, 5 carbon atoms, 6 carbon atoms, 7 carbon atoms, or 8 carbon atoms. For example, “C1-C4 alkyl” means an alkyl group which can be composed of 1 carbon atom, 2 carbon atoms, 3 carbon atoms, or 4 carbon atoms. As used herein, alkyl also includes cases in which the numerical range is unspecified.

The term “alkenyl” means an optionally substituted straight chain or an optionally substituted branch-chain monovalent hydrocarbon group having at least one C═C double bond. The alkenyl group has, but is not limited to, 2-8 carbon atoms, for example, 2-6 carbon atoms or 2-4 carbon atoms. The double bond of these groups can be either cis-form or trans-form conformation, and should be understood to include both isomers. Examples of alkenyl groups include, but are not limited to, vinyl (CH═CH₂), 1-propenyl (CH₂CH═CH₂), isopropenyl (C(CH₃)═CH₂), butenyl, and 1,3-butadienyl, etc. Where an alkenyl as defined herein appears in a numerical range, for example, “C2-C8 alkenyl” means an alkenyl group which can be composed of 2 carbon atoms, 3 carbon atoms, 4 carbon atoms, 5 carbon atoms, 6 carbon atoms, 7 carbon atoms, or 8 carbon atoms. As used herein, alkenyl also includes cases in which the numerical range is unspecified.

The term “alkynyl” means an optionally substituted straight chain or an optionally substituted branch-chain monovalent hydrocarbon group having at least one C≡C triple bond. The alkynyl group has, but is not limited to, 2-8 carbon atoms, for example, 2-6 carbon atoms or 2-4 carbon atoms. Examples of alkynyl groups herein include, but are not limited to, ethynyl, 2-propynyl, 2-butynyl, and 1,3-butyldiynyl, etc. Where an alkynyl as defined herein appears in a numerical range, for example, “C2-C8 alkynyl” means an alkynyl group which can be composed of 2 carbon atoms, 3 carbon atoms, 4 carbon atoms, 5 carbon atoms, 6 carbon atoms, 7 carbon atoms, or 8 carbon atoms. As used herein, alkynyl also includes cases in which the numerical range is unspecified.

The term “cyclic hydrocarbon group” means a non-aromatic carbon-containing ring, including a saturated carbon ring (for example, cycloalkyl) or an unsaturated carbon ring (for example, cycloalkenyl). Carbon rings include a monocyclic carbon ring (having one ring), for example, a monocyclic cycloalkyl; a bicyclic carbon ring (having two rings), for example, a bicyclic cycloalkyl; and a polycyclic carbon ring (having two or more rings). The rings can be in a bridge linked or spiro ring relationship. The carbon ring (for example, cycloalkyl or cycloalkenyl) may have 3 to 8 carbon atoms, for example, 3 to 6 ring-forming carbon atoms or 3 to 5 ring-forming carbon atoms. Cyclic hydrocarbon groups of 3 to 20 carbon atoms can be, for example, cyclic hydrocarbon groups of 3 to 12 carbon atoms, such as cyclopropyl, cyclobutyl, cyclopentyl, cyclohexyl, cyclooctyl, etc.

The term “aryl” refers to any optionally substituted aromatic hydrocarbon group having 6 to 20, for example, 6 to 12 or 6 to 10 ring-forming carbon atoms, which may be a monocyclic aryl, a bicyclic aryl or more cyclic aryl. A bicyclic aryl group or polycyclic aryl group may be a group in which a monocyclic aryl group is fused to another independent ring, such as alicyclic ring, heterocyclic ring, aromatic ring, aromatic heterocyclic ring. A non-limiting example of a monocyclic aryl is a monocyclic aryl group having 6 to 12, 6 to 10 or 6 to 8 ring-forming carbon atoms, such as phenyl. A non-limiting example of a bicyclic aryl is, for example, naphthyl. Non-limiting examples of polycyclic aryl include, for example, phenanthrenyl, anthryl, and azulenyl.

The term “heteroaryl” refers to optionally substituted heteroaryl groups consisting of about 5 to 20, for example, 5 to 12 or 5 to 10 skeletal ring-forming atoms, of which at least one (for example, 1 to 4, 1 to 3, 1 to 2) is a heteroatom. The heteroatom is independently selected from, but not limited to, of the group consisting of oxygen, nitrogen, sulfur, phosphorus, silicon, selenium, and tin. Heteroaryls include monocyclic heteroaryl (having one ring), bicyclic heteroaryl (having two rings) or polycyclic heteroaryl (having two or more rings). In embodiments in which there are two or more heteroatoms in the ring, the two or more heteroatoms may be identical to each other, or some or all of the two or more heteroatoms may be different from each other. A bicyclic heteroaryl group or more polycyclic heteroaryl group may be a group (may collectively, fused ring heteroaryl group) in which a monocyclic heteroaryl group is fused to another independent ring, such as alicyclic ring, heterocyclic ring, aromatic ring, and aromatic heterocyclic ring. Non-limiting examples of heteroaryl groups include, but not limited to, pyrrolyl, furanyl, thienyl, imidazolyl, oxazolyl, pyrazolyl, pyridinyl, pyrimidinyl, pyrazinyl, quinolinyl, isoquinolinyl, tetrazolyl, triazolyl, triazinyl, benzofuranyl, benzothienyl, indolyl, and isoindolyl etc.

The term “heterocyclyl” refers to a non-aromatic heterocyclyl that includes either saturated or unsaturated heterocyclic rings (including unsaturated bonds), which do not have a fully conjugated x-electron system and can be classified as non-aromatic monocyclic ring, fused-polycyclic ring, bridged-ring or spiro-ring systems, in which one or more (for example, 1 to 4, 1 to 3, 1 to 2) of the ring-forming atoms are heteroatoms, such as an oxygen, nitrogen or sulfur atom. A heterocycle ring includes a monocyclic ring (comprising one ring), a bisheterocyclic ring (comprising two bridge-linked rings), a polyheterocyclic ring (comprising two or more bridge-linked rings), and a spirocylic ring. The heterocyclyl may have 3 to 20, for example, 3 to 10, 3 to 8, 4 to 8, 4 to 7, 5 to 8, or 5 to 6 ring-forming atoms. Non-limiting examples of heterocyclyls include: oxyranyl, thiiranyl, aziridinyl, azetidinyl, oxetanyl, thietanyl, tetrahydrofuranyl, pyrrolidinyl, oxazolidinyl, tetrahydropyrazolyl, pyrrolinyl, dihydrofuranyl, dihydrothienyl, piperidyl, tetrahydropyranyl, tetrahydrothionyl, morpholinyl, piperazinyl, dihydropyridyl, tetrahydropyridyl, Dihydropyranyl, tetrahydropyranyl, dihydrothionyl, azepanyl, oxepanyl, thiepanyl, oxa-azabicyclo[2.2.1]heptyl, and azaspiro[3.3]heptyl, etc.

Other group terms herein include: “hydroxyl” referring to the —OH group, “alkylthio” referring to the —SH group, “cyano” referring to the —CN group, and “carboxyl” referring to the —COOH group.

### Compound

The present disclosure provides a compound for a photolithographic medium composition, having a structural formula represented by general formula (11) below

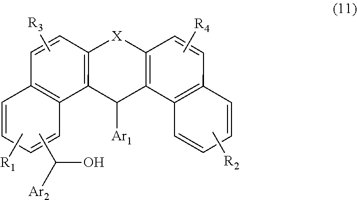

wherein X is a heteroatom, and independently selected from the group consisting of an oxygen atom, a sulfur atom, and a nitrogen atom;
Ar₁ and Ar₂ are the same or different, and each independently selected from the group consisting of a C6-C20 aryl substituted by 0 to 3 R^{A}, a heteroaryl containing 3 to 20 skeletal ring-forming atoms and containing one or more identical or different heteroatoms substituted by 0 to 3 R^{A}, a heterocyclyl containing 3 to 20 skeletal ring-forming atoms and containing one or more identical or different heteroatoms substituted by 0 to 3 R^{A}, and a cyclic hydrocarbon group containing 3 to 20 carbon atoms substituted by 0 to 3 R^{A};
R₁ and R₂ are each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group an ester group;
R₃ and R₄ are each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group;
R^{A} is each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group; and
R¹¹ and R¹² are each independently selected from the group consisting of hydrogen, a C1-C8 alkyl, a C2-C8 alkenyl and a C2-C8 alkynyl.

In one embodiment, the compound has a structure represented by general formula (12) or (13).

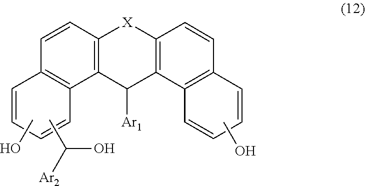

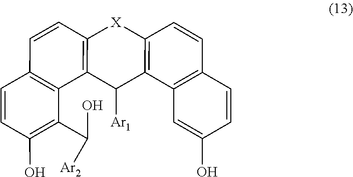

wherein Ar₁, Ar₂, and X are as defined in the formula (11) above, and —C(OH)Ar₂ in formula (12) is located at the ortho position of the hydroxyl group.

In one embodiment, X is an oxygen atom.

It should be noted that, in the present disclosure, Ar₁ and Ar₂ should have some sterically hindered effect in order to form a compound having a single sec-hydroxyl structure.

In one embodiment, Ar₁ and Ar₂ are the same, and independently selected from the group consisting of a C6-C20 aryl substituted by 0 to 3 R^{A}. Preferably, the C6-C20 aryl is selected from the group consisting of phenyl, naphthyl, fluorenyl, anthracenyl, phenanthrenyl, pyrenyl, biphenyl, and triphenyl.

In one embodiment, Ar₁ and Ar₂ are the same, and independently selected from a C3-C20 cyclic hydrocarbon group, for example, C5-C8 cycloalkyl group, such as cyclopentyl, cyclohexyl, cycloheptyl, cyclooctyl, etc, which is substituted by 0 to 3 R^{A}.

In one embodiment, Ar₁ and Ar₂ are the same, and independently selected from a heteroaryl containing 3 to 20 skeletal ring-forming atoms and containing one or more identical or different heteroatoms, for example, heteroaryl containing 5 to 10 skeletal ring-forming atoms, such as pyridinyl, etc, which is substituted by 0 to 3 R^{A}.

The compound can be prepared by reacting the compound of formula A below with one or more Ar—CHO aldehyde compounds (the Ar—CHO compound includes Ar₁—CHO and Ar₂—CHO, wherein Ar₁ and Ar₂ are defined above) in the presence of an acid catalyst.

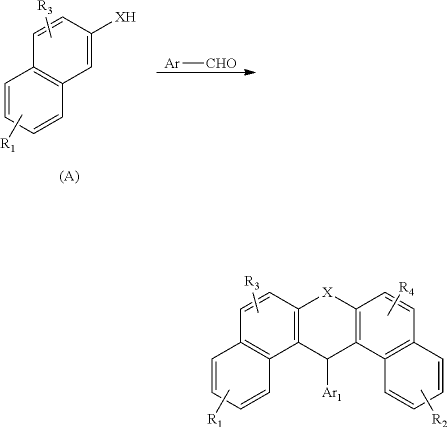

The compounds of formula A can be substituted or non-substituted naphthalene compounds, such as naphthol, dihydroxynaphthalene, dimercaptonaphthalene, naphthylamine, hydroxynaphthylamine, etc. If R₁ or R₂ is a hydroxyl group, the —C(OH)Ar₂ in the resulting compound is usually located at the ortho position of the hydroxyl group.

Dihydroxynaphthalene may include 2,7-dihydroxynaphthalene, 2,6-dihydroxynaphthalene, 1,6-dihydroxynaphthalene, 2,3-dihydroxynaphthalene, and so on. Dimercaptonaphthalene may include 2,7-dimercaptonaphthalene, 2,6-dimercaptonaphthalene, 1,6-dimercaptonaphthalene, 2,3-dimercaptonaphthalene, and so on. Hydroxynaphthyamine may include 6-hydroxynaphthalene-2-amine, 7-hydroxynaphthalene-2-amine, and so on.

Ar—CHO aldehyde compounds may include one or more aldehydes, such as Ar₁—CHO and Ar₂—CHO, wherein Ar₁ and Ar₂ are defined above. For example, it may be benzaldehyde, furfural, hydroxybenzaldehyde (including m-hydroxybenzaldehyde and p-hydroxybenzaldehyde), naphthaldehyde, anthraldehyde, biphenylcarboxaldehyde, pyrenecarboxaldehyde, and so on.

The inventors of the present disclosure found that during the above reaction, Ar—CHO aldehyde compound should be in stoichiometric excess relative to the compound of formula A. For example, 1.1 to 2.0 mol of Ar—CHO aldehyde compound is used for 1 mol of the compound of formula A. If the stoichiometry of the Ar—CHO aldehyde compound is greater than that of compound A, i.e. greater than one equivalent, then in the case of a complete reaction, the products are all compounds having a sec-hydroxyl structure. The inventors of the present disclosure found that if the stoichiometry of Ar—CHO aldehyde compound is less than that of the compound of formula A, and especially if the stoichiometry of Ar—CHO aldehyde compound is less than or equal to 0.5 times that of compound A, it is virtually impossible to obtain a compound having a sec-hydroxyl structure.

The acid catalysts used in the above reaction can be inorganic acids and organic acids. For example, inorganic acids may include hydrochloric acid, sulfuric acid, and organic acids may include p-toluene sulfonic acid, methanesulfonate, acetic acid, benzenesulfonic acid, trifluoromethanesulfonate, etc. Lewis acids such as aluminum chloride and zinc chloride can also be used. The amount of acid catalyst can be 0.001 to 0.1 mol for 1 mol of the compound of formula A.

The above reaction can be carried out by using reaction solvents such as alcohol solvent (such as methanol, ethanol, etc.), ether solvent (such as diethyl ether, cyclopentyl methyl ether, propylene glycol monomethyl ether, ethylene glycol dimethyl ether, etc.), ester (such as propylene glycol methyl ether acetate, ethyl lactate, ethyl acetate, butyl acetate, etc.), halogenated hydrocarbon (such as dichloromethane, chloroform, dichloroethane, etc.), or combination thereof.

The temperature of the above reaction can be selected according to the reaction raw materials and the type of catalyst, and be generally 50-160° C. However, in order to facilitate the formation of the compounds of the present disclosure, it is usually performed at relatively high temperatures, for example, 90-160° C.

After completion of the reaction, the compounds of the present disclosure may be isolated by using techniques known in the art. Alternatively, other reactive raw materials may be directly added to the reaction system to obtain the polymers described below in this disclosure.

### Polymers

On one hand, the present disclosure provides a polymer for a photolithographic medium composition, having a structural unit represented by general formula (1) below

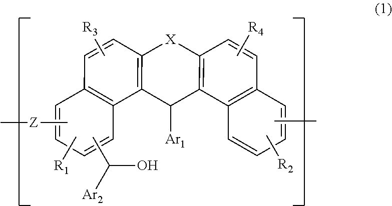

wherein X is a heteroatom, and each independently selected from the group consisting of an oxygen atom, a sulfur atom, and a nitrogen atom;
Ar₁ and Ar₂ are the same or different, and each independently selected from the group consisting of a C6-C20 aryl substituted by 0 to 3 R^{A}, a heteroaryl containing 3 to 20 skeletal ring-forming atoms and containing one or more identical or different heteroatoms substituted by 0 to 3 R^{A}, a heterocyclyl containing 3 to 20 skeletal ring-forming atoms and containing one or more identical or different heteroatoms substituted by 0 to 3 R^{A}, and a cyclic hydrocarbon group containing 3 to 20 carbon atoms substituted by 0 to 3 R^{A};
R₁ and R₂ are each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group;
R₃ and R₄ are each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group;
Z is selected from the group consisting of a single bond, a C1-C10 alkylene substituted by 0 to 3 R^{A}, a C6-C20 arylene substituted by 0 to 3 R^{A}, a C6-C20 aralkylene substituted by 0 to 3 R^{A}, a C4-C20 heteroaralkylene substituted by 0 to 3 R^{A}, and C1-C10 heteroalkylene substituted by 0 to 3 R^{A};
R^{A} is each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group; and
R¹¹ and R¹² are each independently selected from the group consisting of hydrogen, a C1-C8 alkyl, a C2-C8 alkenyl and a C2-C8 alkynyl.

Preferably, the structural unit represented by general formula (1) is the structural unit represented by general formula (2)

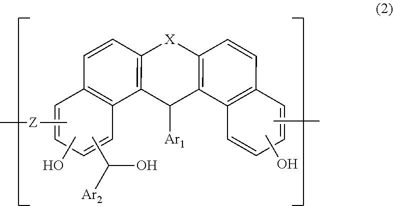

wherein in formula (2), Ar₁, Ar₂, X and Z are as defined in the formula (1) above, and —C(OH)Ar₂ is located at the ortho position of the hydroxyl group.

Preferably, the structural unit represented by general formula (1) is the structural unit represented by general formula (3)

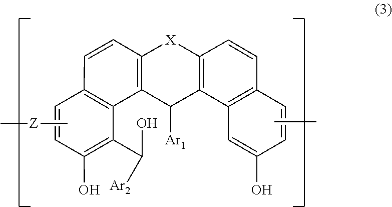

wherein in formula (3), Ar₁, Ar₂, X and Z are as defined in the formula (1) above.

In one embodiment, X is an oxygen atom.

It should be noted that, in the present disclosure, Ar₁ and Ar₂ should have some sterically hindered effect in order to form a polymer having a single sec-hydroxyl structure.

In one embodiment, Ar₁ and Ar₂ are the same, and independently selected from a C6-C20 aryl substituted by 0 to 3 R^{A}. Preferably, C6-C20 aryl is selected from the group consisting of phenyl, naphthyl, fluorenyl, anthracenyl, phenanthrenyl, pyrenyl, biphenyl, and triphenyl.

In one embodiment, Ar₁ and Ar₂ are the same, and independently selected from a C3-C20 cyclic hydrocarbon group, for example, a C5-C8 cycloalkyl group, such as cyclopentyl, cyclohexyl, cycloheptyl, cyclooctyl, etc, which is substituted by 0 to 3 R^{A}.

In one embodiment, Ar₁ and Ar₂ are the same, and independently selected from heteroaryl containing 3 to 20 skeletal ring-forming atoms and containing one or more identical or different heteroatoms, for example, a heteroaryl containing 5 to 10 skeletal ring-forming atoms, such as pyridinyl, etc, which is substituted by 0 to 3 R^{A}.

In one embodiment, Z can be selected from the following structures

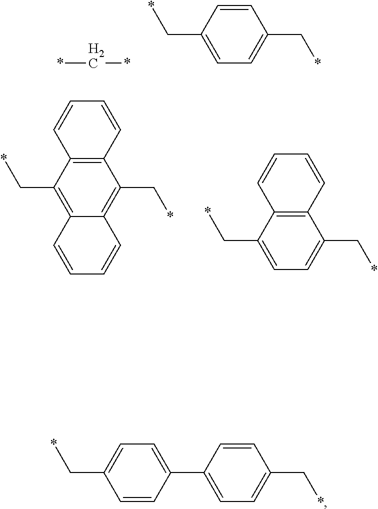

In the above structures, * indicates the connecting position of the above structures.

The polymer can be prepared by reacting the compounds of the present disclosure with monomers having cross-linking reactivity. Alternatively, it is also possible to obtain the polymer of the present disclosure by adding monomers having cross-linking reactivity to the reaction system after obtaining the desired compounds in the process of preparing the compound of the present disclosure as described above.

As monomers having cross-linking reactivity, aldehydes such as formaldehyde and paraformaldehyde, and diol compounds such as p-benzedimethanol, biphenyldimethanol, naphthalenedimethanol, anthracenedimethanol, etc. can be used. The amount of monomers having cross-linking reactivity is 0.5 to 1.5 mol, especially 0.8 to 1.2 mol for 1 mol of the compound of the present disclosure.

The above reaction steps can be carried out in the presence of a catalyst. The catalyst may be an acid catalyst, and inorganic acids and organic acids can be used. For example, inorganic acids such as hydrochloric acid, sulfuric acid, etc., and organic acids such as p-toluene sulfonic acid, acetic acid, benzenesulfonic acid, trifluoromethanesulfonate, etc. can be used as the acid catalysts. Lewis acids such as aluminum chloride and zinc chloride can also be used.

The above reaction can be carried out by using reaction solvents such as ether solvent (such as diethyl ether, cyclopentyl methyl ether, propylene glycol monomethyl ether, ethylene glycol dimethyl ether, etc.), ester (such as propylene glycol methyl ether acetate, ethyl lactate, ethyl acetate, butyl acetate, etc.), halogenated hydrocarbon (such as dichloromethane, chloroform, dichloroethane, etc.), or combination thereof.

As mentioned above, it is also possible to obtain the polymer of the present disclosure by adding monomers having cross-linking reactivity to the reaction system after obtaining the desired compounds in the process of preparing the compound of the present disclosure as described above. Since an acid catalyst and a solvent have been already used in the reaction process for preparing the compounds of the present disclosure, it is not necessary to further add an acid catalyst and a solvent to this process. Therefore, this preparation process is preferable.

In one embodiment, the polymer may have the weight average molecular weight of 500 to 20000 Da, preferably 1000 to 5000 Da. The molecular weight distribution can be 1.1 to 5.0.

The compounds and polymers of the present disclosure contain sec-hydroxyl structure, which can improve their solubility and wettability of the material on the substrate, thereby improving the quality of film formation. In addition, the sec-hydroxyl structure is a reactive group, and can react with crosslinking agent to increase the crosslinking degree of the membrane layer, which in turn can improve the etching resistance of the membrane layer. Therefore, the above-mentioned compounds and polymers of the present disclosure have a high solubility in solvents. In particular, they have very good solubility in propylene glycol monomethyl ether acetate (PGMEA), propylene glycol monomethyl ether (PGME), and cyclohexanone, and exhibits excellent etching resistance.

### Photolithographic Medium Composition

It is generally required in industrial applications that the materials for etching-resistant medium layer should have even better etching resistance. In recent years, several efforts have been made to develop materials for medium layers and to apply these materials to multi-layer stacking processes. However, previous experience has shown that improving the etching resistance of materials (e.g., the pursuit of higher carbon content) often comes at the expense of solubility and film forming ability.

However, the compounds and polymers of the present disclosure maintain a carbon-rich structure (i.e., a polybenzene ring structure) while a sec-hydroxyl structure is introduced into the structure, thereby increasing the polar interaction sites of the material, increasing the mobility of the structure, and contributing to the improvement of the ability to interact with solvents. Thus, the solubility performance of the material is improved. In addition, the sec-hydroxyl structure can form cross-linking sites during the film formation process of the material, which can improve the overall cross-linking density of the material, and consequently ensure the etching resistance. The compounds and the polymers of the present disclosure have excellent performance in the solubility and etching resistance, and the solubility of the compounds and the polymers are optimized while the etching resistance is considered. Thus, the compounds and the polymers are very suitable as the material for an etching-resistant medium layer.

The present disclosure also relates to a photolithographic medium composition that contains an acid generating agent, a crosslinking agent, and a medium material, wherein the medium material is the above-mentioned polymer and/or compound of the present disclosure.

The medium material contained in the photolithographic medium composition of the present disclosure may be above-mentioned polymers and/or compounds of the present disclosure, wherein based on the total weight of the photolithographic medium composition, the medium material is present in an amount of 0.1 to 30 wt %, preferably 2 to 15 wt %, more preferably 3 to 10 wt %.

The photolithographic medium composition of the present disclosure may contain, in addition to the medium material described above, an acid generating agent, a cross-linking agent, a surfactant, and a solvent, etc.

In one embodiment, based on the total weight of the photolithographic medium composition, the acid generating agent is present in an amount of 0.001 to 10 wt %, preferably 0.01 to 5 wt %.

The acid generating agent may comprise a thermal acid generating agent and an optional photo acid generating agent. In one embodiment, as the thermal acid generating agent, either ionic thermal acid generating agent or non-ionic thermal acid generating agent can be used. Ionic thermal acid generating agents include, but are not limited to, sulfonates such as carbocyclylaryl sulfonate and heteroaryl sulfonate, aliphatic sulfonate, benzene sulfonate, triflate, triethylamine dodecyl sulfonate; and ammonium p-toluenesulfonate. Non-ionic thermal acid generating agents include, but are not limited to, p-toluenesulfonic acid, methyl trifluoromethanesulfonate, cyclohexyl trifluoromethanesulfonate, cyclohexyl 2,4,6-triisopropyl benzenesulfonate, 2-nitrobenzyl p-toluenesulfonate, alkyl ester of organic sulfonic acid, benzoin tosylate, 2-nitrobenzyl toluenesulfonate, tris(2,3-dibromopropyl)-1,3,5-triazine-trione, dodecylbenzene sulphonic acid, oxalic acid, phthalic acid, phosphoric acid and camphorsulfonic acid, etc. and salts thereof, and the thermal acid generating agents disclosed in patent U.S. Pat. No. 10,429,737B2. Based on the total weight of the photolithographic medium composition, the content of the thermal acid generating agent is 0.001 to 10 wt %, preferably 0.01 to 5 wt %, and more preferably 0.01 to 3 wt %.

The photo acid generating agent may include, for example, onium salts such as (tetra-t-butyl phenyl)-trifluoromethanesulfonate iodonium salt, triphenyl trifluoro methanesulfonate sulfonium salt, etc.; the compounds containing halogen for photo acid generating agents, such as phenyl tosylate, N-hydroxyl succinimidyl bis(trichloromethyl)-s-triazine, etc.; benzoin trifluoromethanesulfonate type photo acid generating agent; disulfonyl diazomethane type, etc. (onium salts, such as triphenylsulfonium trifluoromethanesulfonate, (p-tert-butoxyphenyl) diphenylsulfonium trifluoromethanesulfonate, tris(p-tert-butoxyphenyl) sulfonium trifluoromethanesulfonate, triphenylsulfonium p-toluenesulfonate; nitrobenzyl derivatives, such as 2-nitrobenzyl-p-toluenesulfonate, 2,6-dinitrobenzyl-p-toluenesulfonate and 2,4-dinitrobenzyl-p-toluenesulfonate; sulfonates, such as 1,2,3-tris(methanesulfonyloxy)benzene, 1,2,3-tris(trifluoromethanesulfonyloxy)benzene, and 1,2,3-tris(p-toluenesulfonyloxy)benzene; diazomethane derivatives, such as bis(phenylsulfonyl)diazomethane, bis(p-toluenesulfonyl) diazomethane; glyoxime derivatives, such as bis-O-(p-toluenesulfonyl)-α-dimethyl glyoxime and bis-O-(n-butanesulfonyl)-α-dimethyl glyoxime; sulfonic acid ester derivatives of N-hydroxylimide compound, such as N-hydroxyl succinimide methanesulfonate, N-hydroxyl succinimide trifluoromethanesulfonate; and triazine compounds containing halogen, such as 2-(4-methoxyphenyl)-4,6-bis(trichloromethyl)-1,3,5-triazine and 2-(4-methoxynaphthalenyl)-4,6-bis(trichloromethyl)-1,3,5-triazine). In one embodiment, based on the total weight of the photolithographic medium composition, the content of the photo acid generating agent is 0 to 10 wt %, preferably 0 to 5 wt %, more preferably 0.01 to 3 wt %.

The photolithographic medium composition of the present disclosure may contain a cross-linking agent. In one embodiment, based on the total weight of the photolithographic medium composition, the amount of cross-linking agent is 0.01 to 10 wt %. The cross-linking agent used in the present disclosure may be glycoluril derivatives, melamine derivatives, biphenol derivatives etc., for example, hexahydroxymethylmelamine, hexamethoxymethylmelamine, hexamethoxyethylmelamine, and so on; tetrahydroxymethylglycoluril, tetramethoxyglycoluril, tetramethoxymethylglycoluril, and so on.

A surfactant may be added to the photolithographic medium composition formed according to the present disclosure. Surfactants may include, for example, polyoxyethylene alkyl ethers such as polyoxyethylene stearyl ether, polyoxyethylene lauryl (dodecyl) ether, polyoxyethylene hexadecyl ether, polyoxyethylene oleyl ether, etc.; polyoxyethylene alkyl-aryl ethers such as polyoxyethylene octylphenol ether, polyoxyethylene nonylphenol ether etc.; polyoxyethylene, polyoxypropylene block polymer, sorbitan monolaurate, sorbitan monopalmitate (hexadecanoate), sorbitan monostearate, sorbitan monooleate (9-octadecenoate), polyoxyethylene sorbitan monolaurate, sorbitan trioleate, sorbitan tristearate; polyoxyethylene sorbitan monopalmitate (hexadecanoate), polyoxyethylene sorbitan trioleate, polyoxyethylene sorbitan monostearate, polyoxyethylene sorbitan monooleate (9-octadecenoate), polyoxyethylene sorbitan tristearate, etc. In one embodiment, based on the total weight of the photolithographic medium composition, the content of the surfactant is 0 to 20 wt %, more preferably 0.0001 to 5 wt %.

Solvents for the photolithographic medium compositions formed according to the present disclosure include: single solvents such as alcohols, esters, ethers, cyclic ketones, etc., or mixed solvents thereof. Solvents include, but are not limited to: methyl ethyl ketone, cyclopentanone, cyclohexanone, ethylene glycol monomethyl ether, ethylene glycol monoethyl ether, methyl cellosolve acetate, ethyl cellosolve acetate, diethylene glycol monomethyl ether, diethylene glycol monoethyl ether, propylene glycol, propylene glycol monomethyl ether, propylene glycol monomethyl ether acetate, propylene glycol propyl ether acetate, toluene, xylene, ethyl 2-hydroxypropanoate, methyl 2-hydroxy-3-methyl butyrate, ethyl pyruvate, ethyl acetate, butyl acetate, ethyl lactate, butyl lactate, methyl 3-methoxypropionate, ethyl 3-methoxypropionate, ethyl 3-ethoxypropionate, methyl 3-ethoxypropionate, methyl pyruvate, N,N-dimethylformamide, dimethyl sulfoxide, N-methylpyrrolidone etc. In one embodiment, based on the total weight of the photolithographic medium composition, the content of the solvent is 70 to 99 wt %, and more usually 85 to 99 wt %.

The present disclosure also relates to a photolithographic medium layer formed from above-mentioned photolithographic medium composition of the present disclosure. The method for forming the medium layer is not particularly limited, and the medium layer can be formed by applying the photolithographic medium composition on a substrate through a method known to those skilled in the art such as coating methods or printing methods (known as spin coating, screen printing, etc.), then volatilizing the organic solvents. After film formation, the cross-linking reaction can be promoted by baking and the like. In one embodiment, the baking temperature may be 80 to 400° C., in particular 200 to 400° C.

Hereinafter, examples are provided to further illustrate the disclosure.

### Synthesis Example 1

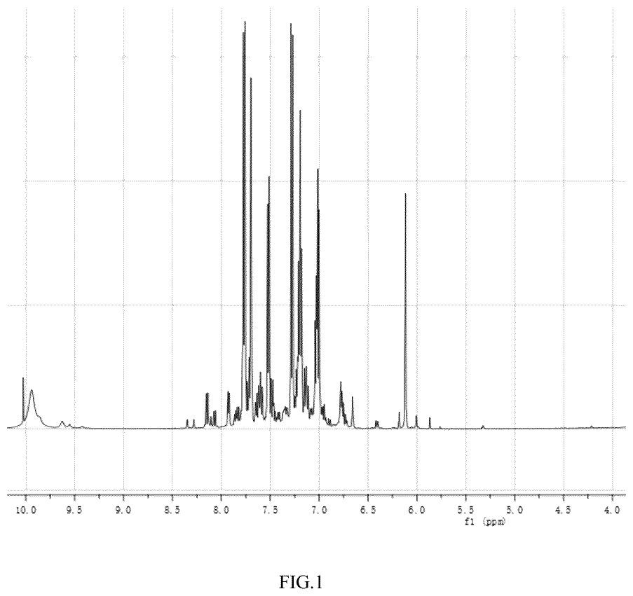

To a 200 ml reaction flask with magnetic agitation and condensed reflux, 16 g (0.1 mol) of 2,7-dihydroxynaphthalene, 15.9 g (0.15 mol) of benzaldehyde and 100 ml of cyclopentyl methyl ether were added, stirred at 60° C. for 10 minutes to completely dissolve; then 0.95 g of p-toluene sulfonic acid and 10 ml of glacial acetic acid were added and heated to reflux and reacting for 24 hours to obtain intermediate a-1. The chemical structure was confirmed by 500 MHz ¹H-NMR, the spectrum was shown in FIG. 1, δ(a-1) (ppm, DMSO, TMS): 9.52-9.91 (—OH), 6.80-8.22 (Ph-H, PH₂-CH—OH), 6.70 (Ph₂-CH), 6.12 (Ph₂-CH—OH).

After Cooling to room temperature, 1.5 g of paraformaldehyde was added, and heated again to reflux and reacting for 6 hours. At the end of the reaction, the product was precipitated with 500 ml of n-hexane and filtered. It was washed sequentially with deionized water and n-hexane and dried in vacuum oven at 50° C., and the target polymer A-1 was obtained. The product had the weight average molecular weight of 3400 Da and Mw/Mn of 2.51, determined by gel chromatography.

The chemical structure was confirmed by 500 MHz ¹H-NMR, δ(A-1) (ppm, DMSO, TMS): 9.52-9.91 (—OH), 6.80-8.22 (Ph-H, Ph₂-CH—OH), 6.70 (Ph₂-CH), 6.12 (Ph₂-CH—OH), 4.41-4.88 (CH₂).

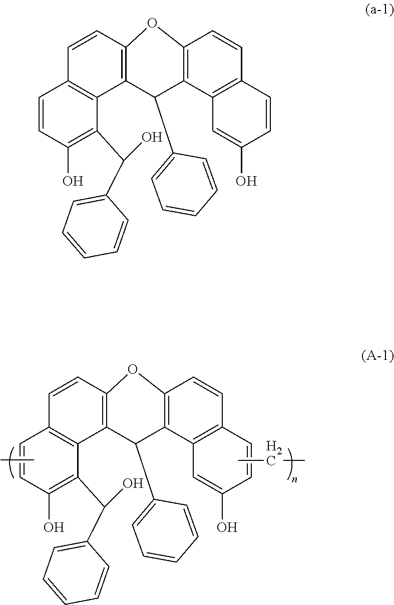

### Synthesis Example 2

Intermediate a-2 and polymer A-2 were obtained by the same operation as in Synthesis example 1, except that naphthaldehyde was used to replace benzaldehyde. The polymer had the weight average molecular weight of 1500 Da, and Mw/Mn of 2.63.

The chemical structure of intermediate a-2 was confirmed by 500 MHz ¹H-NMR, δ(a-2) (ppm, DMSO, TMS): 9.30-9.91 (—OH), 6.80-8.22 (Ph-H, PH₂-CH—OH), 6.70 (Ph₂-CH), 6.30 (Ph₂-CH—OH).

The chemical structure of polymer A-2 was confirmed by 500 MHz ¹H-NMR, δ(A-2) (ppm, DMSO, TMS): 9.30-9.91 (—OH), 6.80-8.22 (Ph-H, Ph₂-CH—OH), 6.70 (Ph₂-CH), 6.30 (Ph₂-CH—OH), 4.03-4.71 (CH₂).

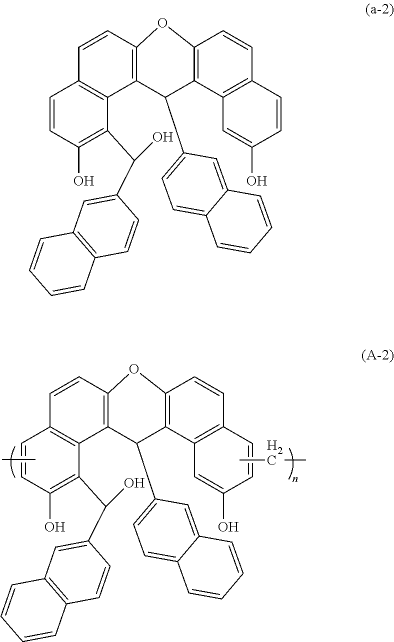

### Synthesis Example 3

Polymer A-3 was obtained by the same operation as in Synthesis example 1, except that p-benzedimethanol was used to replace paraformaldehyde. Polymer A-3 had the weight average molecular weight of 2700 Da and Mw/Mn of 2.45.

The chemical structure was confirmed by 500 MHz ¹H-NMR, δ(ppm, DMSO, TMS): 9.30-9.91 (—OH), 6.81-8.20 (Ph-H, Ph₂-CH—OH), 6.76 (Ph₂-CH), 6.14 (Ph₂-CH—OH), 4.13-4.53 (CH₂).

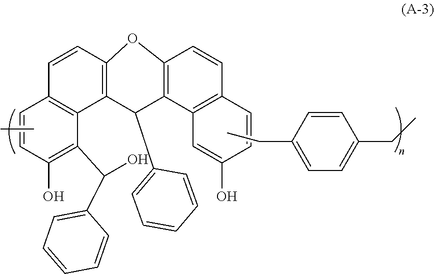

### Synthesis Example 4

Intermediate a-4 and polymer A-4 were obtained by the same operation as in Synthesis example 3, except that m-hydroxybenzaldehyde was used to replace benzaldehyde. Polymer A-4 had the weight average molecular weight of 4400 Da and Mw/Mn of 1.92.

The chemical structure of intermediate a-4 was confirmed by 500 MHz ¹H-NMR, 8 (a-4) (ppm, DMSO, TMS): 9.32-9.91 (—OH), 6.40-8.22 (Ph-H, PH₂-CH—OH), 6.74 (Ph₂-CH), 6.03 (Ph₂-CH—OH).

The chemical structure of polymer A-4 was confirmed by 500 MHz ¹H-NMR, δ(A-4) (ppm, DMSO, TMS): 9.30-9.91 (—OH), 6.40-8.20 (Ph-H, Ph₂-CH—OH), 6.74 (Ph₂-CH), 6.03 (Ph₂-CH—OH), 4.26-4.63 (CH₂).

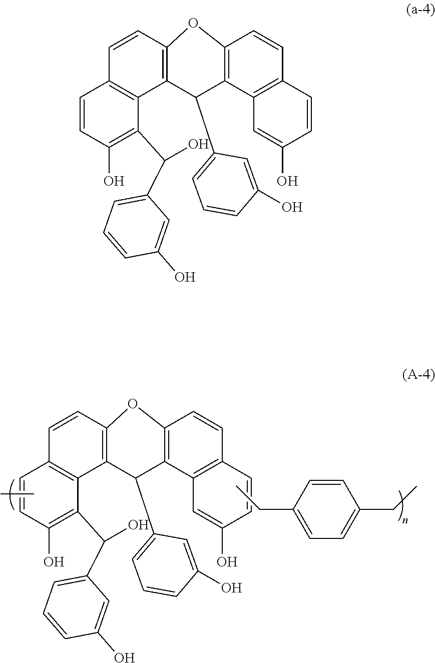

### Synthesis Example 5

Intermediate a-5 and polymer A-5 were obtained by the same operation as in Synthesis example 1, except that 2-formylpyridine was used to replace benzaldehyde. Polymer A-5 had the weight average molecular weight of 2300 Da and Mw/Mn of 1.89.

The chemical structure of intermediate a-5 was confirmed by 500 MHz ¹H-NMR, δ(a-5) (ppm, DMSO, TMS): 9.06-10.21 (—OH), 6.58-8.58 (Ph-H, >CH—OH), 6.75 (Ph₂-CH), 6.30 (>CH—OH).

The chemical structure of polymer A-5 was confirmed by 500 MHz ¹H-NMR, δ(A-5) (ppm, DMSO, TMS): 9.06-10.31 (—OH), 6.58-8.87 (Ph-H, >CH—OH), 6.71 (Ph₂-CH), 6.31 (>CH—OH), 4.40-5.21 (CH₂).

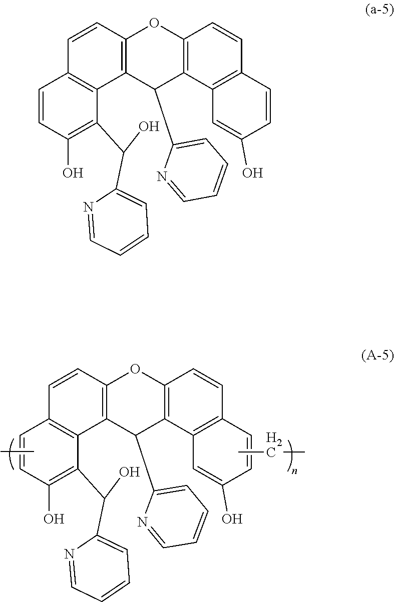

### Synthesis Example 6

Intermediate a-6 and polymer A-6 were obtained by the same operation as in Synthesis example 1, except that cyclohexanecarboxaldehyde was used to replace benzaldehyde. Polymer A-6 had the weight average molecular weight of 2400 Da and Mw/Mn of 2.01.

The chemical structure of intermediate a-6 was confirmed by 500 MHz ¹H-NMR, δ(a-6) (ppm, DMSO, TMS): 9.03-10.09 (—OH), 6.58-8.23 (Ph-H, >CH—OH), 4.59 (Ph₂-CH), 5.04 (>CH—OH), 1.05-2.05 (cyclohexyl-H).

The chemical structure of polymer A-6 was confirmed by 500 MHz ¹H-NMR, δ(A-6) (ppm, DMSO, TMS): 9.07-10.01 (—OH), 6.56-8.28 (Ph-H, >CH—OH), 4.60 (Ph₂-CH), 5.05 (>CH—OH), 4.01-5.41 (CH₂), 1.01-2.20 (cyclohexyl-H).

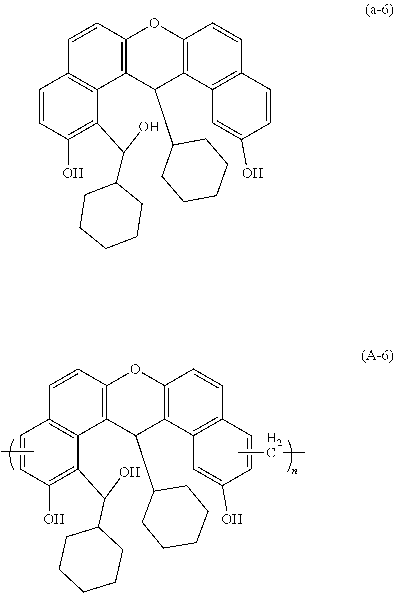

### Comparative Synthesis Example 1

To a 200 ml reaction flask with magnetic agitation and condensed reflux, 14.4 g (0.1 mol) of 2-hydroxynaphthalene, 3 g of paraformaldehyde, and 100 ml of cyclopentyl methyl ether were added, stirred at 60° C. for 10 minutes to completely dissolve. 0.95 g of p-toluene sulfonic acid was then added and heated to reflux and reacting for 24 hours. At the end of the reaction, the product was precipitated with 500 ml of n-hexane and filtered. It was washed sequentially with deionized water and n-hexane and dried in vacuum oven at 50° C., and the target polymer B-1 was obtained. The product had the weight average molecular weight of 3300 Da, and Mw/Mn of 2.32, determined by gel chromatography.

The chemical structure was confirmed by 500 MHz ¹H-NMR, δ(ppm, DMSO, TMS): 9.52-9.92 (—OH), 7.00-8.22 (Ph-H), 4.30-4.68 (CH₂).

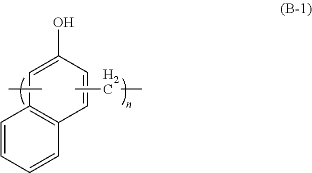

### Comparative Synthesis Example 2

To a 200 ml reaction flask with magnetic agitation and condensed reflux, 16 g (0.1 mol) of 2,7-dihydroxynaphthalene, 5.3 g (0.05 mol) of benzaldehyde and 100 ml of cyclopentyl methyl ether were added, stirred at 60° C. for 10 minutes to completely dissolve. then 0.95 g of p-toluene sulfonic acid was added and heated to reflux and reacting for 24 hours. After Cooling to room temperature, 1.5 g of paraformaldehyde, and 10 ml of glacial acetic acid were added, and heated again to reflux and reacting for 6 hours. At the end of the reaction, the product was precipitated with 500 ml of n-hexane and filtered. It was washed sequentially with deionized water and n-hexane and dried in vacuum oven at 50° C., and the target polymer B-2 was obtained. The product had the weight average molecular weight of 1200 Da, and Mw/Mn of 1.95, determined by gel chromatography.

The chemical structure was confirmed by 500 MHz ¹H-NMR, δ(B-2) (ppm, DMSO, TMS): 9.52-9.91 (—OH), 6.80-8.22 (Ph-H), 6.70 (Ph₂-CH), 4.40-4.78 (CH₂).

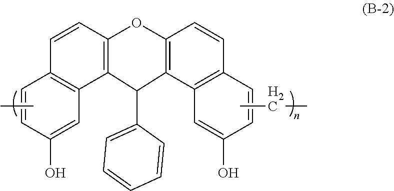

### Preparation and Performance Testing of Etching Resistant Coating Compositions

### Example 1

### (1) Solubility Evaluation

Polymer A-1 was respectively dissolved in 100 g of propylene glycol monomethyl ether acetate, propylene glycol monomethyl ether and cyclohexanone at 25° C. The maximum dissolution amount of the polymer was recorded. If the maximum dissolution amount is 20 g or more, the solubility is considered as “excellent”; if the maximum dissolution amount is between 10 g and 20 g, the solubility is considered as “good”; and, if the maximum dissolution amount is less than 10 g, the solubility is considered as “poor”.

### (2) Optical Test

0.4 g of polymer A-1 obtained in Synthesis example 1 was dissolved in 10 g of mixed solution of propylene glycol monomethyl ether acetate and propylene glycol monomethyl ether with a volume ratio of 7:3.8 mg of p-toluene sulfonic acid, 0.08 g of acid crosslinking agent Powderlink 1174, and 2 mg of surfactant polyoxyethylene dehydrated sorbitan trioleate were added. The solution was well mixed and filtered with a 0.22 μm filter to obtain the photolithographic medium composition.

The composition was spin coated on a silicon wafer at 1500 rpm and heated and baked at 250° C. for 60 seconds to form a thin film. The thickness of the thin film was measured by spectroscopic ellipsometer, and the refractive index n and the extinction coefficient k at 193 nm were investigated.

### (3) Evaluation of Etching Resistance

The obtained thin films were respectively etched for 60 seconds at a power of 300 W, a flow rate of 40 sccm and a pressure of 8 mtorr in CF₄ plasma gas, and for 30 seconds at a power of 50 W, a flow rate of 8 sccm and a pressure of 8 mTorr in O₂ plasma gas. The thickness of the thin films was measured by spectroscopic ellipsometer to calculate the thickness change value of thin films. Thus, the etching rate of the obtained thin films in two plasma gases was calculated according to equation 1-1.

Etching rate (nm/min)=the thickness change value of film (nm)/time (min) (equation 1-1)

All data are aggregated and presented in Table 1-1.

### Example 2

Except for the replacement of A-1 by A-2, the same composition preparation and testing methods as in Example 1 were used.

### Example 3

Except for the replacement of A-1 by A-3, the same composition preparation and testing methods as in Example 1 were used.

### Example 4

Except for the replacement of A-1 by A-4, the same composition preparation and testing methods as in Example 1 were used.

### Example 5

Except for the replacement of A-1 by A-5, the same composition preparation and testing methods as in Example 1 were used.

### Example 6

Except for the replacement of A-1 by A-6, the same composition preparation and testing methods as in Example 1 were used.

### Example 7

Except for the replacement of A-1 by a-1, the same composition preparation and testing methods as in Example 1 were used.

### Example 8

Except for the replacement of A-1 by a-2, the same composition preparation and testing methods as in Example 1 were used.

### Example 9

Except for the replacement of A-1 by a-4, the same composition preparation and testing methods as in Example 1 were used.

### Example 10

Except for the replacement of A-1 by a-5, the same composition preparation and testing methods as in Example 1 were used.

### Example 11

Except for the replacement of A-1 by a-6, the same composition preparation and testing methods as in Example 1 were used.

### Comparative Example 1

Except for the replacement of A-1 by B-1, the same composition preparation and testing methods as in Example 1 were used.

### Comparative Example 2

Except for the replacement of A-1 by B-2, the same composition preparation and testing methods as in Example 1 were used.

TABLE 1-1

| | | | | | | | CF₄ | O₂ |
|---|---|---|---|---|---|---|---|---|
| | | | | | | | etching | etching |
| | | solubility | | | Refractive | Extinction | rate | rate |
| Examples | materials | PMA | PGME | cyclohexanone | index | coefficient | nm/min | nm/min |
| E1 | A-1 | good | excellent | excellent | 1.56 | 0.56 | 12 | 26 |
| E2 | A-2 | good | good | good | 1.31 | 0.44 | 10 | 23 |
| E3 | A-3 | excellent | excellent | excellent | 1.55 | 0.62 | 12 | 27 |
| E4 | A-4 | excellent | excellent | excellent | 1.40 | 0.55 | 12 | 25 |
| E5 | A-5 | good | excellent | excellent | 1.56 | 0.42 | 11 | 25 |
| E6 | A-6 | excellent | excellent | excellent | 1.51 | 0.24 | 13 | 29 |
| E7 | a-1 | good | excellent | excellent | 1.57 | 0.59 | 12 | 28 |
| E8 | a-2 | good | good | good | 1.37 | 0.40 | 11 | 26 |
| E9 | a-4 | excellent | excellent | excellent | 1.45 | 0.50 | 12 | 27 |
| E10 | a-5 | good | excellent | excellent | 1.51 | 0.51 | 12 | 27 |
| E11 | a-6 | excellent | excellent | excellent | 1.52 | 0.24 | 13 | 29 |
| CE1 | B-1 | excellent | excellent | excellent | 1.36 | 0.31 | 13 | 33 |
| CE2 | B-2 | poor | good | poor | 1.55 | 0.54 | 12 | 28 |

As can be seen from the statistical results in Table 1-1, under the conditions of the blended composition solutions, the polymers A-1 to A-6 used in Examples 1 to 6 all have superior solubility performance, as compared with the polymer B-2 used in Comparative example 2. This suggests that the introduction of sec-hydroxyl structure has a positive effect on improving the 5 solubility of the polymer, which ensures a better processing window for such compositions.

Similarly, as can be seen from Table 1-1, under the testing conditions designed in the present experiment, Examples 1-11 had remarkably more excellent performance of etching resistance compared with Comparative example 1. As compared with Comparative example 2, Examples 1-5 and 7-10 were equivalent or advantageous in terms of etching rate. As compared 10 with Examples 1 to 5, Examples 7 to 10 prepared from small molecule materials were slightly inferior in terms of etching resistance. It was considered that this is related to the crosslinking degree and crosslinking structure of the film materials after crosslinking.

In the foregoing, the present disclosure has been described incorporating preferred embodiments, but these embodiments are illustrative only and are intended for illustration purposes. On this basis, it is possible to make various substitutions and improvements to this application, all of which fall within the scope of protection of this application.

1. A polymer for a photolithographic medium composition, having a structural unit represented by general formula (2) below 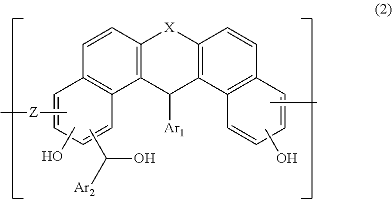
wherein X is a heteroatom, and each independently selected from the group consisting of an oxygen atom, a sulfur atom, and a nitrogen atom;
Ar₁ and Ar₂ are the same or different, and each independently selected from the group consisting of a C6-C20 aryl substituted by 0 to 3 R^{A}, a heteroaryl containing 3 to 20 skeletal ring-forming atoms and containing one or more identical or different heteroatoms substituted by 0 to 3 R^{A}, a heterocyclyl containing 3 to 20 skeletal ring-forming atoms and containing one or more identical or different heteroatoms substituted by 0 to 3 R^{A}, and a cyclic hydrocarbon group containing 3 to 20 carbon atoms substituted by 0 to 3 R^{A}; and —C(OH)Ar₂ is located at the ortho position of the hydroxyl group;
Z is selected from the group consisting of a single bond, a C1-C10 alkylene substituted by 0 to 3 R^{A}, a C6-C20 arylene substituted by 0 to 3 R^{A}, a C6-C20 aralkylene substituted by 0 to 3 R^{A}, a C4-C20 heteroaralkylene substituted by 0 to 3 R^{A}, and a C1-C10 heteroalkylene substituted by 0 to 3 R^{A};
R^{A} is each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group; and
R¹¹ and R¹² are each independently selected from the group consisting of hydrogen, a C1-C8 alkyl, a C2-C8 alkenyl and a C2-C8 alkynyl;
the weight average molecular weight of the polymer is 500 to 20000 Da; and the molecular weight distribution of the polymer is 1.1 to 5.0.

2. (canceled)

3. The polymer according to claim 1, wherein the structural unit represented by general formula (2) is the structural unit represented by general formula (3) 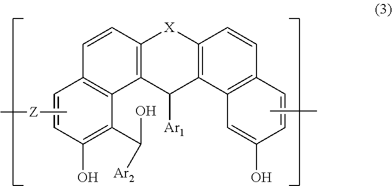
wherein Ar₁, Ar₂, X and Z are as defined in claim 1.

4. The polymer according to claim 1, wherein X is an oxygen atom.

5. The polymer according to claim 1, wherein Ar₁ and Ar₂ are the same, and independently selected from a C6-C20 aryl substituted by 0 to 3 R^{A}.

6. The polymer according to claim 5, wherein the C6-C20 aryl is selected from the group consisting of phenyl, naphthyl, fluorenyl, anthracenyl, phenanthrenyl, pyrenyl, biphenyl, and triphenyl.

7. The polymer according to claim 1, wherein Z is selected from the following structures

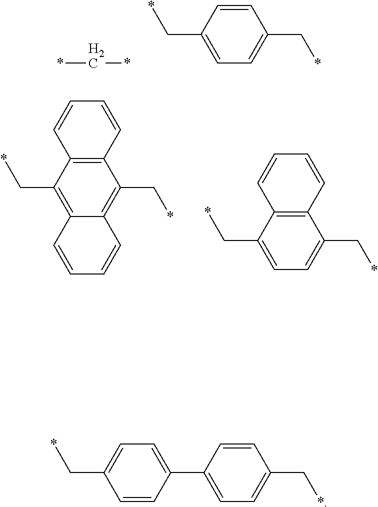

8. The polymer according to claim 1, wherein the weight average molecular weight of the polymer is 1000 to 5000 Da.

9. A compound for a photolithographic medium composition, having a structure represented by general formula (12) below 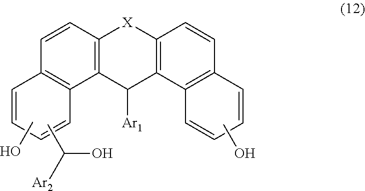
wherein X is a heteroatom, and independently selected from the group consisting of an oxygen atom, a sulfur atom, and a nitrogen atom;
Ar₁ and Ar₂ are the same or different, and each independently selected from the group consisting of a C6-C20 aryl substituted by 0 to 3 R^{A}, a heteroaryl containing 3 to 20 skeletal ring-forming atoms and containing one or more identical or different heteroatoms substituted by 0 to 3 R^{A}, a heterocyclyl containing 3 to 20 skeletal ring-forming atoms and containing one or more identical or different heteroatoms substituted by 0 to 3 R^{A}, and a cyclic hydrocarbon group containing 3 to 20 carbon atoms substituted by 0 to 3 R^{A};
R^{A} is each independently selected from the group consisting of hydrogen, halogen, cyano, a C1-C8 alkyl, a C2-C8 alkenyl, a C2-C8 alkynyl, —OR¹¹, —SR¹¹, —NR¹¹R¹², an ether group and an ester group; and
R¹¹ and R¹² are each independently selected from the group consisting of hydrogen, a C1-C8 alkyl, a C2-C8 alkenyl and a C2-C8 alkynyl; and
in general formula (12), —C(OH)Ar² is located at the ortho position of the hydroxyl group.

10. The compound according to claim 9, wherein the structure represented by general formula (12) is the structure represented by general formula (13) 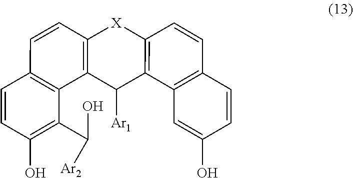
wherein Ar₁, Ar₂, X are as defined in claim 9.

11. The compound according to claim 9, wherein X is an oxygen atom.

12. The compound according to claim 9, wherein Ar₁ and Ar₂ are the same, and independently selected from a C6-C20 aryl substituted by 0 to 3 R^{A}.

13. A photolithographic medium composition comprising an acid generating agent, a crosslinking agent, and a medium material, wherein the medium material is the polymer of claim 1.

14. The photolithographic medium composition according to claim 13, wherein based on the total weight of the photolithographic medium composition, the amount of the medium material is 0.1 to 30 wt %;
based on the total weight of the photolithographic medium composition, the amount of cross-linking agent is 0.01 to 10 wt %; and
based on the total weight of the photolithographic medium composition, the amount of acid generating agent is 0.001 to 10 wt %.

15. The photolithographic medium composition according to claim 1, wherein the acid generating agent comprises a thermal acid generating agent and an optional photo acid generating agent,
wherein based on the total weight of the photolithographic medium composition, the amount of the thermal acid generating agent is 0.001 to 10 wt %; and
based on the total weight of the photolithographic medium composition, the amount of the photo acid generating agent is 0 to 10 wt %.

16. The photolithographic medium composition according to claim 13, further comprising a surfactant and a solvent.

17. The photolithographic medium composition according to claim 16, wherein based on the total weight of the photolithographic medium composition, the amount of the surfactant is 0 to 20 wt %; and
based on the total weight of the photolithographic medium composition, the amount of the solvent is 70 to 99 wt %.

18. A photolithographic medium layer formed from the photolithographic medium composition of claim 13.

19. The compound according to claim 12, the C6-C20 aryl is selected from the group consisting of phenyl, naphthyl, fluorenyl, anthracenyl, phenanthrenyl, pyrenyl, biphenyl, and triphenyl.

20. A photolithographic medium composition comprising an acid generating agent, a crosslinking agent, and a medium material, wherein the medium material is the compound of claim 9.

21. The photolithographic medium composition according to claim 14, wherein based on the total weight of the photolithographic medium composition, the amount of the medium material is 2 to 15 wt %.

22. The photolithographic medium composition according to claim 14, wherein based on the total weight of the photolithographic medium composition, the amount of the medium material is 3 to 10 wt %.

23. The photolithographic medium composition according to claim 15, wherein based on the total weight of the photolithographic medium composition, the amount of the thermal acid generating agent is 0.01 to 5 wt %.

24. The photolithographic medium composition according to claim 15, wherein based on the total weight of the photolithographic medium composition, the amount of the thermal acid generating agent is 0.01 to 3 wt %.

25. The photolithographic medium composition according to claim 15, wherein based on the total weight of the photolithographic medium composition, the amount of the photo acid generating agent is preferably 0 to 5 wt %.

26. The photolithographic medium composition according to claim 15, wherein based on the total weight of the photolithographic medium composition, the amount of the photo acid generating agent is 0.01 to 3 wt %.

27. The photolithographic medium composition according to claim 17, wherein based on the total weight of the photolithographic medium composition, the amount of the surfactant is 0.0001 to 5 wt %.

28. The photolithographic medium composition according to claim 17, wherein based on the total weight of the photolithographic medium composition, the amount of the solvent is 85 to 99 wt %.
